(19) United States
(12) Patent Application Publication
(10) Pub. No.: US 2022/0221547 A1
(43) Pub. Date: Jul. 14, 2022

(54) Method and System of Location Monitoring and Tracking

(71) Applicants: Steven L Wenrich, Quitman, TX (US); Rick Thomas Murphy, Colorado Springs, CO (US)

(72) Inventors: Steven L Wenrich, Quitman, TX (US); Rick Thomas Murphy, Colorado Springs, CO (US)

(21) Appl. No.: 17/617,550

(22) PCT Filed: Jun. 8, 2019
(86) PCT No.: PCT/US2019/036192

§ 371 Date: Dec. 8, 2021

(51) Int. Cl. G01S 5/00; G01S 5/02

(52) U.S. Cl. CPC G01S 5/0027; G01S 5/0295; G16H 40/63; G01S 2205/10; G01S 5/0269

(57) ABSTRACT

A system and method of tracking and monitoring the location of devices for preventing theft and unauthorized use; and the tracking and monitoring of worn devices with sensors for monitoring persons requiring assistance and/or supervision.

### BACKGROUND OF THE INVENTION

### Cross-Reference to Related Applications

Not Applicable

### STATEMENT REGARDING FEDERALLY SPONSORED RESEARCH OR DEVELOPMENT

Not Applicable.

### Reference to Sequence Listing, a Table, or a Computer Program Listing Compact Disc Appendix

Not Applicable.

### FIELD OF THE INVENTION

This invention refers in general to a method and system of monitoring a person or object's location, reporting its removal from a desired location, and continuing to track its changing location to assist in recovery. Specifically, this invention utilizes multiple layers of electronic communication technology to monitor a device with electronic communication connections and those connection's signal levels to determine proximity within a predetermined area, alert multiple parties to removal from the predetermined area, and continue to track, and report locations outside of the predetermined area to assist in recovery.

### BACKGROUND OF THE INVENTION

Electronic communications capabilities are becoming ubiquitous. Many public access locations have wireless local-area networks (WLAN) providing Internet connectivity to patrons/customers. Most modern WLANs are based on standards in the Institute of Electrical and Electronics Engineer (IEEE) 802.11 family, which are commercially known as ‘Wi-Fi’® communications.

Another communication standard, IEEE 802.15 known as Bluetooth®, though prevalent, it is often thought of as a short-range technology. This is not the case. A class 1 Bluetooth device can transmit at 100 mW producing a standard range of approximately 100 meters which is comparable to that of an 802.11b WLAN device.

Cellular networks require a Subscriber Identity Module (SIM) to identify a device's broadband processor to the network. A SIM card includes an Integrated Circuit Card Identifier (ICC-ID) and an International Mobile Subscriber Identity (IMSI) which are the authentication information to identify the carrier and customer to be billed for cellular tower access.

The Global Positioning System (GPS) is a satellite-based radio-navigation system owned by the United States Government. GPS provides geographical (geo) location and time information to GPS receivers with an accuracy of less than a meter. However, that resolution of location can only be determined when there is access to four or more satellites. GPS does not require a user to transmit data and operates independent of any cellular or computer network reception.

Many businesses have taken advantage of technology familiarization to streamline data collection or improve customer experiences. For example, doctor's offices are notorious for collecting extensive amounts of data about a patient's current symptoms, general health, medical history, family history, etc. Rather than writing copious notes that must later be interpreted and entered into a records system, the use of laptops, electronic tablets, portable notepads, etc. has become more common.

Forms can be completed electronically making poor handwriting a non-issue. Pre-defined choices make it easier for a patient/user to answer, and standardize responses making data more useful to interpret. This same scenario can be generalized to situations where a business may want to collect any type of data or may want to provide information to a customer/user which may not be appropriate for general broadcast from a public monitor due to confidentiality, relevancy, or simply to allow time shifting to accommodate the customer/user's needs and schedule.

However, the drawback is that portable electronic devices are ‘portable.’ A device can easily be mislaid and go unnoticed due to its small size; a distracted patient may unintentionally carry the device from the office; or worse, a device may intentionally be stolen by an unscrupulous person. This is impactful when the device may contain someone's confidential information. It is particularly harmful if the device contains medical information, where improper exposure can be a HIPAA violation carrying fines of $100 to $50,000 per record and possible criminal charges.

### BRIEF DESCRIPTION OF THE DRAWINGS

FIG. 1 illustrates a physician's office with electronic communications equipment for an implementation in accordance with an exemplary embodiment of the innovation.

FIG. 2 illustrates a large open business environment with electronic communications equipment in accordance with an exemplary embodiment of the innovation.

FIG. 3 shows an explanation of triangulation procedures as utilized in accordance with an exemplary embodiment of the innovation.

FIG. 4 shows an explanation of distance determinization procedures as utilized in accordance with an exemplary embodiment of the innovation.

FIG. 5 shows a flowchart of primary operations for a monitoring routine implemented in a monitored and tracked device in accordance with an exemplary embodiment of the innovation.

FIG. 6 shows a flowchart of primary operations for a monitoring routine implemented in a wearable device for monitoring and tracking locations and associated data in accordance with an exemplary embodiment of the innovation.

FIG. 7 shows a flowchart of primary operations for a centralized server routine of a monitoring system for monitoring and tracking locations and associated data in accordance with an exemplary embodiment of the innovation.

FIG. 8 shows a diagram of communication channel associations for the implementation of monitoring and tracking of locations and associated data in accordance with an exemplary embodiment of the innovation.

FIG. 9 shows a diagram of communication and sensor requirement for a wearable smart device utilized for implementation of monitoring and tracking of locations and associated data in accordance with an exemplary embodiment of the innovation.

### DETAILED DESCRIPTION OF THE PREFERRED EMBODIMENTS

This innovation describes a system for monitoring electronics to prevent unauthorized removal from a predefined location and tracks the electronics for retrieval and protection of contents therein. Additionally, this system can be expanded to the monitoring and tracking of people for a variety of purposes.

In a situation such as the doctor's office described above, office staff, e.g. a receptionist (designated the local administrator or system operator) may provide a tablet, smart phone, or laptop (the monitored device) to a patient (a user) upon checking in for an appointment. The user is expected to remain in the waiting area (a first zone) to complete the information request on the monitored device and return it to the local administrator. Occasionally, the user may take the monitored device with them to the treatment area (a second zone) but is still expected to return the monitored device to the local administrator before leaving.

For this illustration's purpose, assume the waiting area is an open room with a door leading to treatment areas, and a different door exiting from the office into the ‘rest of the world,’ (a third zone). The treatment area is comprised of a corridor with multiple exam rooms accessible from the corridor. The treatment area has no other means of leaving the building other than returning to the waiting area. The waiting area, the first zone, is a ‘safe zone.’ The treatment area, the second zone, is a ‘caution zone.’ Everywhere else, the third zone, is a ‘danger zone.’ In this scenario we may wish to monitor the safe zone more closely, since a device could transition from there directly to a danger zone. We may not need to monitor the caution zone as closely since the scenario does not allow transition directly to the danger zone directly without first returning to the safe zone.

In the described embodiments three zones are described as being a safe zone, a caution zone, and a danger zone. These coincide with three levels of monitoring having various levels of concern, monitoring activities, and interactions or responses. One skilled in the art would appreciate that there can be more or less zones, and distinct characteristics could apply to each zone or that particular actions may have varying effects depending on the specific zone in which they occur.

A monitoring program on the monitored device (referred to hereafter as the ‘tracked device’ or ‘device’ for simplicity) regularly checks the location of the device to verify/determine the current zone, respond to changes in state or functionality of the device and retrieves and processes messages from a central tracker (the central system or server monitoring all tracked devices/users and providing notifications to a designated location or device monitored by the administrator(s), the receiving device and/or monitoring person, interchangeably referenced as the local administrator or system operator.)

The device then takes appropriate action upon transition, or anticipated transition between zones, or other state changes. Depending on the precision of location monitoring, a device may be able to predict transitions within and/or between zones from sensory information, e.g. direction of movement and/or current location.

While the device is in the safe zone, and is fully operational, it will continue to monitor for changes thorough background process unnoticed by the user. This can be accomplished by utilizing Bluetooth beacons to roughly triangulate the devices position in the area. In another embodiment, the detection of one or more beacons may be enough to identify that the device is in a particular zone, and location within the area is irrelevant.

If the device is taken to the treatment area (transitions from safe zone to caution zone), the central server will see the change, and in response, the central server may be configured to send a notification to the local administrator that the device is leaving a safe area. In another embodiment cellular tower signals may be utilized in place of Bluetooth beacons.

In another embodiment GPS satellites may be utilized for location, and zone determination. In another embodiment a plurality of the above embodiments may be combined for location determination, where the plurality of determination methods is prioritized by, but not limited to, one or more of the following factors: equipment cost, signal availability, acquisition timing, battery capabilities.

By notifying the local administrator that a device is moving to the treatment area, an administrator can better keep mental track of device locations and/or the patients/users. Additionally, the patient is reminded to return the device to the receptionist upon leaving the office. This reminder can be a message appearing on the device, a vibration alert, or a sound alert. The options may be configurable by the system administrator during system setup and configuration.

Once the patient moves into the caution zone, the ping frequency may be adjusted. In one embodiment, the ping frequency may be reduced, thus creating less communications traffic on the local computer network because the caution zone, while out of sight of the local administrator, is sufficiently secured against device loss because there is no means of exiting the building without first returning through the safe zone. Additionally, beacons do not have to be present in every room because it is not possible to leave the area without returning through the corridor, and the corridor's beacon will trigger message transition to the central server.

In another embodiment, the ping frequency may be increased because the caution zone is out-of-sight of the local administrator and there is an increased opportunity, or at least a perceived opportunity, for tampering, improper use, or attempts at theft. Additionally, the zone may not be physically contained as described in the example and so increasing the ping frequency could allow earlier detection of, and a quicker response to any improper device activity.

While beacons are not required throughout a location, they must be situated to identify a device's current location to the level of precision desired. In one embodiment location precision may not be necessary beyond identifying position within a defined physical area. (e.g. identifying the device is within a specific room.) As an example, a single beacon which can be received from anywhere within a room but cannot be received from outside of the room would be sufficient for identifying if a device is or is not within the room.

In another embodiment location determination may require greater precision such as identifying movement to predict a zone transition before it actually occurs. Having a plurality of signals along with the location of each signal's orientation allows triangulation of a device to be calculated.

The precision of the location of the signal orientation and signal's travel time determine the precision of accuracy in determining device location. Once a device location is determinable, a plurality of determinations can be used to determine travel, speed, direction, etc. through basic vector mathematics, the details of which are not within the scope of claims for the current innovation.

In another embodiment the device may connect to different Wi-Fi signals and verify device location by the ability to access the unique Wi-Fi signals, which is, by design, unreachable from other zones. It is important that the ping time not be lowered to a rate that would allow a user to leave the caution zone, and travel through the safe zone, reaching the danger zone before a ping initiates and identifies the location change.

At least two pings should occur before a device can be moved from the closest physical location in the caution zone to the first entry in the danger zone. This prevents a perfectly timed abrupt power down (i.e. pulling battery or blocking the antenna) which may allow the device's monitor program to send a ping indicating a power state change request. The central server would not detect the device's communication loss for two full ping cycles.

In another embodiment, the ping frequency may be increased to ensure sufficient pings occur in the corridor. The single beacon can thus be used to establish the farthest distance the device was moved down the corridor before it was moved into one of the exam rooms. If the farthest distance can be established, then comparing it with the known distance from the beacon to the exam room doors allows deduction as to which room was entered. Once this information is known, the last known location of the device can be updated in the central computer, and the ping frequency may be lowered to reduce communication traffic.

A drawback to lowering the ping frequency is that device movement from one room to another during the period of lower frequency may result in the relocation not being detected. In the described scenario subsequent relocation is considered sufficiently unlikely enough to offset the risk of missing a relocation when compared to the advantages of lowering the ping frequency.

If the device is eventually returned to the local administrator, the relocation may be insignificant, depending on the purpose of tracking the data. If the device was abandoned in the caution zone, or an attempt was made to breach the device security (i.e. power-down, Wi-Fi change, etc.) the local administrator may be notified of an incorrect ‘last known location.’

Once the patient is ready to leave the treatment area, their movement into the corridor may be detected, but would not trigger a zone change until the patient returned through the door to the waiting room where different beacons, and/or the Wi-Fi change would trigger a zone change to the central server. The central server would then send a notification to the local administrator that a device has returned to the waiting area.

The patient may just be returning to the waiting area but is more likely leaving the office upon completing their visit. The notified local administrator can be ready to receive the device and retrieve the information contained thereon, then clear confidential data, or otherwise reset it the device to recharge, or await use by the next patient.

If the patient is distracted or forgetful, and continues out the door with the device, the triangulation of the beacons in the waiting room should detect the device coming within proximity of the door, and issue a ping to the central server of a zone change from safe zone, to a caution zone (a small area in front of the door.) The device, upon the zone change detection will send a ping to the central server which will notify the local administrator.

If the patient continues out the door, the device will detect the beacon outside the door, and ping the central server with a zone change from caution to danger. The central server alerts the local administrator and sends an alert message to the device. The device monitor, upon receiving an alert message, activates the cellular network and GPS capabilities of the device. It then turns on the alarm (audible alert), turns on the device vibrations (physical alert), and begins flushing the alert on the screen (visual alert).

After a pause, configurable by the system administrator during system setup and configuration, the device retrieves the GPS location, logs the time and location locally, then uses any available connection to notify the central server of the current location. The device then (in case they were suppressed) again turns on the alarm (audible alert), device vibrations (physical alert), and flashes the alert on the screen (visual alert). This process will repeat until the device is returned or power expires.

The system administrator, during system setup and configuration can also preselect a number of times to alert before data clean routines are executed which remove any confidential data from the system. Alternatively, the central system can send a wipe data message at any time and remotely remove confidential data regardless of the zone of current location. This may be used as a way for the local administrator to clear data that has been retrieved and processed upon a user's return of a device.

In another embodiment, rather than using multiple Bluetooth beacons to triangulate location within an area, the device may simply identify the presence or absence of a communication signal such as a Bluetooth beacon or a Wi-Fi to verify a particular zone. Additionally, the signal strength of a single communication signal may provide a crude, but sufficient position proximity.

One or more beacons may be attenuated by doors, windows, furniture, etc. such that signal strength of one or more signals may provide proximity information. In the preferred embodiment Bluetooth LE (Low Energy) beacons powered by connection to location wiring, rather than batteries allows for more stable and predictable signals.

Triangulation is well known from GPS applications. Recent advances in electronic sensor detection have allowed similar applications on a much smaller scale, such as the single room/area presented here. Bluetooth LE protocols provides three channels for advertising packet broadcast. While one skilled in the arts would appreciate the existence and applicability of other protocols and beacon-like communication devices, this protocol and its characteristics will be used through this description for reference simplicity.

Beacons, such as those described here do not offer connections, nor do they have location intelligence. They are like lighthouses transmitting signals around. Beacons usually transmit their UUID (Universal Unique Identifier), major and minor values, or their namespace and instance IDs (Identification). Configuration of the system requires each beacon be mapped to a physical coordinate.

In the example herein, locations are identified by latitude (lat.) and longitude (long.) fixed to a signed 3-digit whole number with 5 decimal places, resulting in resolution to approximately 1-meter resolution. This allows distances to be represented in the same packet structure by setting the lat. to +000.00000 and the long. to the actual distance in meters. This is because the actual coordinate ±000.0000, ±000.00000 is in the southern Atlantic, and sufficiently far enough from anything of relevance that the specific location is of no interest for our purposes.

While RSSI (Received Signal Strength Indicator) values may vary by distance and maximum broadcasting power, the values identified can be used to determine relative positioning, and then scaled to match known physical distances. Distances can be mapped from a single beacon when a corridor or narrow room limits locations down to essentially a single dimension. Scaling does not assist in resolving calculations to physical values. Kalman filters are a common solution that utilizes the history of measurements to dampen irregularities in transmitted signals and electronic noise.

In another example, the innovations can be applied to a large open area with no change to the system, by only applying changes to the configuration data. Bluetooth is commonly considered a short-range technology. But class 1 Bluetooth devices transmit at 100 mW, (100 milliwatts) producing a standard range of approximately 100 meters, approximately equal to that of an 802.11b WLAN device.

By placing four class 1 Bluetooth LE beacons at the edges of the position to be secured and spacing two 802.11b WLAN devices evenly in the center of the area, triangulation of any three beacons should yield approximately the same results and identify if the device is in the safe zone located within the boundaries established within the beacons. Connection can be to either of the Wi-Fi access points. One skilled in the arts would appreciate that different sized areas may have more or less beacons and/or Wi-Fi access points for an implement in accordance with the innovation.

If the device is outside of the boundaries, then depending on the actual size of the area, compared with the actual signal range, triangulation may still be possible if at least three beacons are identified. If the location is calculatable, it can be confirmed to be inside of the safe zone, or out of the safe zone, but still accessible to the communication signals, which would be the caution zone.

If only two beacons are identified, then triangulation is not possible, and calculations will identify two possible locations. However, one of the two locations would be inside of the safe zone, and can be ignored, because that location would also have resulted in connection to a third and possibly a fourth beacon. By process of elimination, the other possibility must be the actual location, and is definitely outside of the safe zone. The caution zone may be defined by the distance outside of the safe zone the device can travel and still reliably communicate with one of the two Wi-Fi access points.

In another embodiment, a smart device is utilized to monitor a person rather than a device location. This may have application in tracking children, elderly who may occasionally get disoriented or may suffer from memory loss. This implementation utilizes a similar, but modified monitor to what is described above. Such a system is referred to as the person monitor rather than the device monitor. The central system would be substantially the same.

The smart device (referred to hereinafter simply as the device), would include a plurality of the following: Bluetooth, Wi-Fi, cellular, GPS, and/or biological (bio) monitoring. The smart device may have bio sensors for bio monitoring and may have a user input component which may be as simple as one or two dedicated buttons, or as complex as voice recognition. The device should also detect when it is connected to a charging station, though input components may also serve this function.

In monitoring a person, the specific coordinates or room location is not as important. The desire is to have greater range, allowing flexibility and freedom for the individual person while ensuring help/assistance, if needed, can quickly be provided. GPS monitoring is more prevalent, but Bluetooth and Wi-Fi are still necessary because of the inability to reliably connect to GPS within enclosed structures. Additionally, it is simply unnecessary to have such precise tracking. Knowing the general location or address were a person is located may be sufficient for many applications.

For person monitoring ping frequencies may be much lower. In the embodiment described here, ping frequency is set to once every fifteen-minutes for the safe zone, once every five-minutes for the caution zone, and once every two minutes for the danger zone. A predefined special mode (shower time) is set as 30 minutes, and the devices is presumed to be charged each night and should be retrieved no later than 8:00 AM each day. These values are completely arbitrary and are selected here for demonstration purposes only.

More complex input components would allow for different values, or even changing values each day depending on the circumstances. The values may be altered during configuration and setup, or may be regularly altered by a system administrator, or possibly by the local administrator via an application, or means beyond the scope of this specification. The variables presented here are to show how customization may be implemented in an embodiment. It is not the intent of this specification to be all encompassing of the possibilities or fully demonstrate every possibility that may be implementable.

The ‘safe zone’ is identified by a recognized Bluetooth beacon or Wi-Fi that has been previously identified for the user during configuration. In an alternative embodiment more than one safe zone may be identified. For instance, the monitored person may have a Bluetooth beacon at their home which allows them to be in a safe zone anywhere in the house, or out in a garden or on a porch.

Another safe zone may be declared at a local community center, church, or restaurant frequented by the monitored person. The ‘caution zone’ is identified by a geo fence which ideally would encompass all safe zones to allow the monitored person to reach a safe zone without entering a danger zone. The ‘danger zone’ is identified as any position outside of the established geo fence.

An input method may be established that allows the monitored person to be marked as ‘in a safe zone’ regardless of actual location. Doing this for a designated period of time would allow monitoring to automatically resume if not renewed, preventing instances where memory lapse results in days of unintentional non-monitoring.

For instance, the monitored person is in a safe zone because she is visiting her son's house and he is taking care of her and providing any transportation/travel, etc. until approximately 4:00 PM Friday. Around noon on Friday, she decides to extend the visit until 4:00 PM Sunday, so the ‘in a safe zone’ designation may be extended. This would allow the monitored person to still utilize their device, which provides inputs for quick access to emergency assistance, monitoring of bio stats, etc.

In another embodiment, a person may be marked as safe and OK for an extended period to disable monitoring temporarily. For example, the monitored person is going to the hospital for at least 6 days to have knee replacement surgery and physical therapy/rehabilitation. The hospital does not allow the device as it will interfere with IV and injections sites, monitoring equipment, etc. Leaving the device at home on the charger will trigger an alert if not worn by 8:00 AM the next morning. Turning the device off will trigger an alert for lack of monitoring.

In person monitoring, notifications and alerts may be sent to an email or phone number (via SMS message) previously identified during configuration. Rather than a single local administrator, a plurality of email addresses or phone numbers may be included, to be sequentially, or simultaneously notified or alerted. The device may also include inputs that allow the monitored person to summon assistance by pressing a dedicated button that may connect them with an ambulance, or police, etc.

In another embodiment, multiple parties may be contacted differently depending on the circumstances. For instance, one embodiment may notify a single ‘neighbor,’ or ‘local friend’ for a specific notification such as ‘No bio data detected.’ The intention being for someone locally to check and verify the condition of the monitored person whom may have simply forgotten to wear their device. One embodiment may notify ‘all children’ of a change to the caution zone so they are aware that the person is ‘getting out and around.’ In another embodiment, a mother may be notified that her diabetic child, down the street at the playground, is experiencing low blood sugar.

The person monitor starts by listening for a Bluetooth beacon or Wi-Fi signal. If one was found, and it was recognized as a ‘safe zone,’ then the current zone is recorded as a safe zone, and last location is set to the beacon or Wi-Fi's physical location. This may be a description rather than a Lat.|Long. since specific location does not need to be as detailed. Next the device looks for bio signals.

But, if a beacon or Wi-Fi was not detected or recognized, the device activates the cellular network and GPS. Note these are left deactivated if not specifically needed to conserve battery and/or cost. If the GPS location is detected, the location is set to the GPS location. If GPS fails to determine a location, then the device can be assumed to be in an enclosed structure.

It can further be assumed that the location and zone have not changed. This could be because changing locations would result in changes to communication signals which would have recorded a new location. The person must still be at the previous location and being in an enclosed environment has blocked GPS until returning outdoors. If the previous location was identified by a recognized beacon or Wi-Fi signal, it would have to be a ‘safe zone.’ But the beacon may not be detectable at the moment due to interference or a temporary power outage. The position cannot change without a new signal becoming visible, or GPS being detected. If the zone has change, determine the new ping frequency for the current zone. Next the device looks for bio signals.

If bio signals are detected, determined if they are within an acceptable range. If so, then log all stats locally and send a ping to central system. If bio signals are detected or are found to be indicative of a health issue, then set current zone to ‘danger zone,’ and send ping to central system. If bio signals are not detected, then see if the device is on the charger.

If it is not on the charger, wait a predetermined amount of time to allow the removed device to be placed on the charger. No more than 3-5 minutes, but this is a configurable value during setup. If the device is placed on the charger during the wait time, then determine the anticipated retrieval time.

In one embodiment this may be dynamically entered by input devices. In another embodiment the anticipated retrieval time may be determined from past history or inferences, i.e. the monitored person usually goes to bed around 11:00 PM and places the device on the charger. It is currently 10:49 PM, which is within the acceptable tolerance, so it is assumed that the anticipated retrieval time will be 6:00 AM based on previous settings.

In another embodiment, the monitor may simply continue sending pings to the central server indicating an ‘all-clear’ type of message without notifying the system of the special mode. This could be an option that allows some level of privacy to be maintained by not broadcasting normal daily routines. Instead, daily routines are detected and identified in the wearable device rather than at the central server. The wearable device could also log all data for later analysis with, for instance, a health professional. Alternatively, the wearable device may only keep a running log of, for instance, the last 24 hours to be accessible/shareable in the event of an incident.

Special modes allow activity and bio stats to alter or go off-line for a certain amount of time without raising an alarm to the system. An example may be that the device is removed to take a shower and will be returned to normal state within 15 minutes. If the device does not return to normal state within the prescribed time, then an alarm can be triggered, and the special state information transmitted to determine what may have occurred and what assistance may be required.

If bio signals are not detected, and the device is on the charger, then has the anticipated retrieval time passed? If not, then set current zone to safe zone, set the bio stats to null values, and send the page. The fact that the zone is set to safe means no alert is raised. If the bio signals are not detected, and the device is on the charger and the anticipated retrieval time has passed, then set the zone to danger, set the bio stats to null values, and send the page. This will send an alert to the local administrator. In an alternate embodiment, one or more additional zones may be established, or alerts may be configured to designate potential medical emergency situations such as when bio stats are not detected as described above.

A device with additional features, examples include, but are not limited to water-proofing, motion sensors, and/or temperature sensors, could detect sudden and excessive momentum over expected thresholds which could signify emergency situations such as a slip and fall in a shower, or an increase in body temperature indicating over-heating or exertion. One skilled in the art would be able to identify additional sensors and/or the conditions that could be detected from individual or combination readings which could be further confirmed or predicted by locations.

### DETAILED DESCRIPTION OF THE DRAWINGS

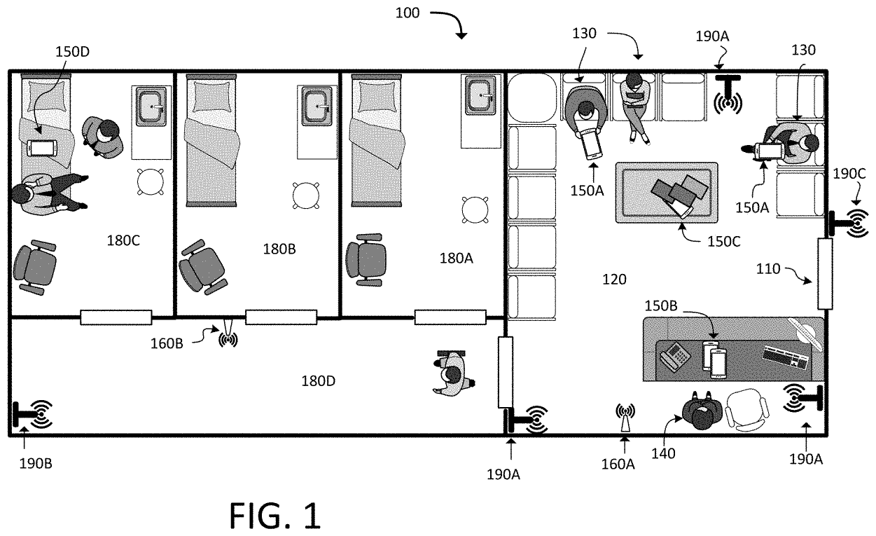

FIG. 1 illustrates a physician's office with electronic communications equipment for an implementation in accordance with an exemplary embodiment of the innovation. The doctor's office (100) is organized into three zones. The first zone is the waiting area (120), which is designated a ‘safe zone.’ The second zone is the treatment area (180), which is designated a ‘caution zone.’ The third zone is anywhere else, which would be outside of the office (not labeled) and is designated a ‘danger zone.’

As a patient/user (130) enters the office (100) through the exterior door (110) the receptionist/administrator (140) provides them one of the monitored devices (150). Users (130) sit in the waiting area (120) and use their assigned device (150A) to answer a questionnaire, take a survey, review their personal information, or otherwise communicate with the physician's staff. Users (130) may also use the devices (150) to access the Internet via the Wi-Fi access point (160).

The devices (150) monitor the environment in background processes, separate from interactions with the user (130). The devices (150) receive signals from Wi-Fi access points (160), which can confirm a device's location as being in the waiting room (120), or in the treatment area (180) depending on which Wi-Fi access point (160A, 160B) transmitted the received signal. The devices (150) also receive signals from one or more beacons (190) and can determine an approximate distance between the device and a beacon (190).

In a location where there are at least three beacons (190A) with known locations, such as the waiting area (120) illustrated, a device (150) can determine its location, and if movement occurs, the device (150) can also determine the direction, and velocity. Small movements may indicate device usage by a user, which can be confirmed by other device interactions with the user. But lack of movement may indicate the device is not in use and may have been discarded (150C) rather than being returned to the administrator (140).

As patients/users (130) transition to the treatment area (180), they may finish with the device (150) and return it to the administrator (140) for any processing and recharging (150B) before being used for another patient/user (130). As a user (130) moves from the waiting area (120) to the treatment area (180), they may take the device (150D). The device can determine a change in location by determining the direction of movement. The device can also determine a change in location by a change in Wi-Fi access point (160A, 160B). Additionally, the device can determine a change in location by receiving a signal from a different beacon (190B) as the user enters the treatment area corridor (180D).

The user may enter the first treatment room (180A), the second treatment room (180B), or the third treatment room (180C). By calculating the distance to the beacon (190B), a device (150D) may determine its location (180C), even if it loses the signal from the beacon (190B) and can report its location to the administrator (140). As a user (130) leaves a treatment room (180A-C) and enters the treatment area corridor (180D), the device (150D) can report to the administrator (140) and alert the user (130) to remind them to return the device (150) to the administrator (140) on the way out of the office (100).

If the user (130) passes the administrator (140) with the device (150) and approaches closer than a specific distance of the exterior door (110), an additional reminder, in the form of a more urgent alert may be provided to the user (130) and the administrator (140). If the alert is not heeded, then as the user (130) moves from the waiting area (120) and exits the doctor's office (100), the device can determine the change in location and take actions, such as raising alarms, locking the device, erasing sensitive information, etc.

When leaving the doctor's office (100), the device can determine a change in location by determining the direction of movement through the waiting area (120). The device can also determine a loss of signal from the Wi-Fi access points (160). Additionally, the device can determine a change in location by receiving a signal from a different beacon (190C) as the user exits the exterior door (110), or by the absence of a recognized communications signal.

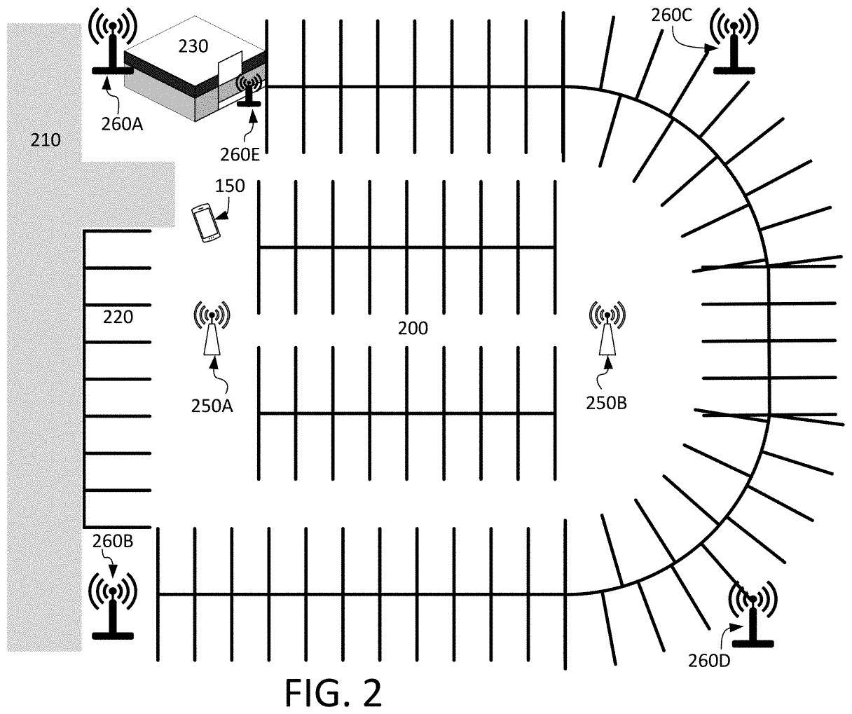

FIG. 2 illustrates a large open business environment with electronic communications equipment in accordance with an exemplary embodiment of the innovation. The example shown is a car dealership consisting of a showroom (230) and an inventory lot (200). In this embodiment there are, again, three zones. The first zone is comprised of the lot (200) and the showroom (230), which is designated a ‘safe zone.’ The second zone, which is designated the ‘caution zone’ is roughly defined as the customer parking area (220), and any area outside of the lot, but still within the immediate area. The third zone, which is labeled the ‘danger zone,’ consist of the street (210), and anywhere else outside of the immediate area.

As a customer/user (not shown) enters from the street (210), they will park their vehicle in the customer parking area (220). The customer is met by a salesman or enters the showroom (230) where they may receive a tracked device (150). The device (150) connects to the local network via Wi-Fi access points (250) and can be used to communicate with the business staff to receive assistance or may provide details regarding the business' inventory on the lot (200) or in the showroom (230).

The lot (200) is delineated by beacons (260) situated on the perimeters. The device (150) monitors the environment in background processes, separate from user interactions. The device (150) receive signals from Wi-Fi access points (250), which can confirm a device's general location depending on which Wi-Fi access point (250A, 250B) transmitted the received signal, and the signal strength. The devices (150) also receive signals from one or more beacons (260) and can determine an approximate distance between the device and a beacon (260).

When a device is toward the back of the lot (200) it may reduce the frequency of location checking. This can be determined by triangulation on three or more beacon (260) signals. But if only two signals can be received, those from beacons 260C and 260D, and the access point in the back of the lot (250B) is visible, then it may be possible to determine the location by triangulation of the three signals. In the alternative, it may be assumed that failure to receive the other two signals (260A, 260B) is due to distance, and the device must be in the caution zone beyond the perimeter delineated by the received signals (260C, 260D). Similar logic would apply to other perimeters.

When a device is toward the front of the lot (200) it may increase the frequency of location checking because there is a greater risk of a customer leaving with the device (150). This again can be determined by triangulation on three or more beacon (260) signals. There is an additional beacon (260E) near the door to the showroom (230) ensuring three signals should be received in the general area.

As described above, the access points (250) may be used to triangulate location. The primary purpose of the additional beacon (260E) near the door to the showroom (230) is that distance from this beacon (260E) can confirm a device's (150) entry into the showroom (230), explaining why there may be no received signals, and confirming the location. Tracking the device (150) can allow the device to remind a customer if the device determines it is approaching the customer parking area (220), or to take appropriate response if the device were to leave the lot (200).

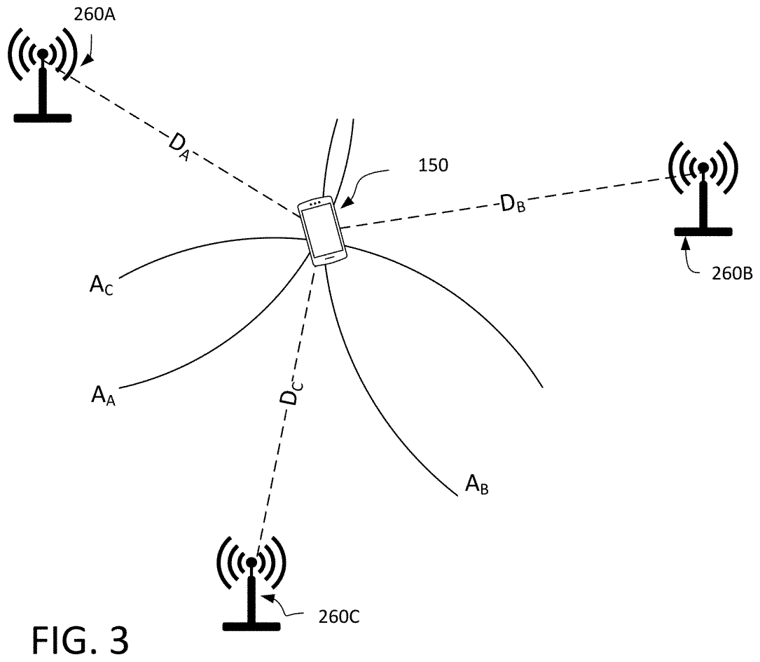

FIG. 3 shows an explanation of triangulation procedures as utilized in accordance with an exemplary embodiment of the innovation. The location of a device (150) can be determined when three or more signals are received, and their distances determined from the signal strength. One skilled in the art would appreciate that given some assumptions, it may be possible to determine location from less signals, and more signals may produce greater accuracy. However, these issues, while relevant to the current innovation, are generally beyond the scope of what is presented here.

Signal strength is affected by distance between a receiver and a transmitter. It is also affected by other environmental characteristics. In FIG. 3, the device (150) receives a signal from a first transmitter (260A) and determines a first distance (D_{A}) from the signal strength. This first distance (D_{A}) establishes the location of the device (150) to be on the perimeter of a circle centered on the first transmitter (260A) and having a radius of the first distance (D_{A}), a portion of which is represented by a first are (A_{A}).

Similarly, from a second transmitter (260B) a second distance (D_{B}) can be determined and is represented by a second arc (A_{B}). Finally, a third transmitter (260C) establishes a third distance (D_{C}), represented by a third arc (A_{C}). If the location of the transmitters (260A, 260B. 260C) are known and the distances (D_{A}, D_{B}, D_{C}) are accurate, then the arcs (A_{A}, A_{B}, A_{C}) should intersect at a single point, which is the location of the device (150).

In the event that the three arcs do not intersect, environmental characteristics affecting the determination of distances can be countered by proportionally adjusting the distances until the arcs intersect establishing a location. This assumes the effects are substantially similar for all of the signals. One skilled in the arts would appreciate that other methods may be applied to reduce error in location determination.

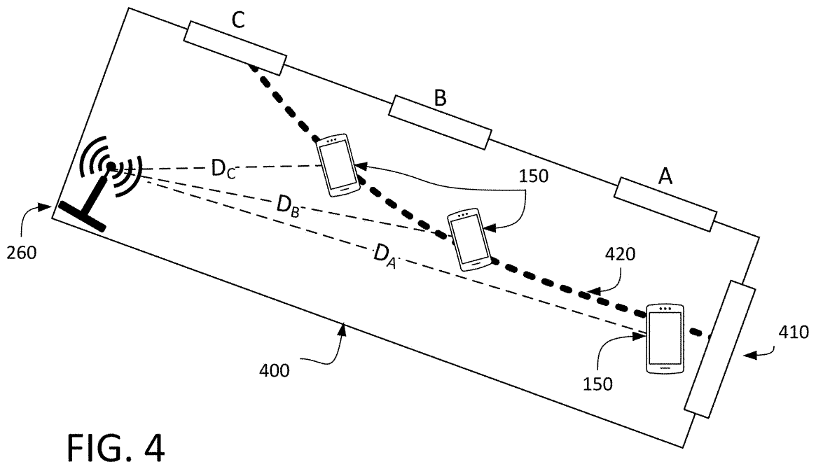

FIG. 4 shows an explanation of distance determinization procedures as utilized in accordance with an exemplary embodiment of the innovation. In some situations, the location of a device (150) can be determined from a single beacon (260) by determining distances. In the corridor (400) a single beacon (260) is used to determine if a device (150) enters one of three doors (A, B, C). As the device crosses the entrance (410) and follows a typical path (420) to a door the distances (D_{A}, D_{B}, D_{C}) are repeatedly determined until the signal of the beacon (260) is lost by passing through a door. If the signal is not lost, then the location is determined to be within the corridor.

If the distances from the beacon (260) to each of the doors (A, B, C) is known, the selected door can be assessed by the last distance calculated. If the last distance (D_{C}) is less than the distance to doors A and B, but greater than the distance to door C, the location can be assumed to be through door C. If the last distance (D_{B}) is less than the distance to door A, but greater than the distance to doors B and C, the location can be assumed to be through door B. If the last distance (D_{A}) is greater than the distance to doors A, B, and C, the location can be assumed to be through door A.

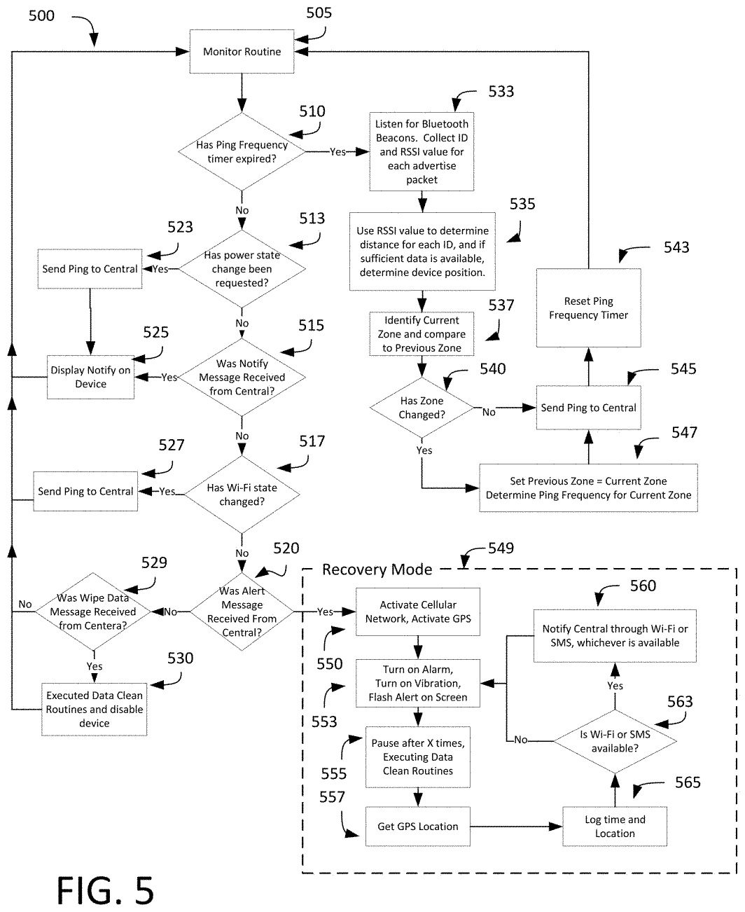

FIG. 5 shows a flowchart of primary operations for a monitoring routine implemented in a monitored and tracked device in accordance with an exemplary embodiment of the innovation. One skilled in the arts would appreciate that the flow/order of operations and the division of operations described herein are not specific, nor all inclusive. Other embodiment may have additional operations or less operations.

The flowchart (500) is for a monitoring routine (505) which is operating in the background of a computing device capable of multi-threaded operations. The monitoring routine (505), which may also be known as a thread, may be timer-based, rotation-based, or event-based activation. Depending on the activation method, it may be necessary to determine if the ping frequency timer has expired (510). If it has not expired, then determine any other reason for activation.

Has a power state change been requested (513)? A user should not change the power state, so a change may be an indication of suspicious activity. Therefore, send a ping to the central server (523), and display a notification on the device (525). The notification may warn the user and confirm they want to attempt the power state change, or it may warn, and reject the attempt to change the power state unless an authorization code is entered. The authorization code may be a passcode known by the administrator which allows normally prevented activities without raising alarm within the system.

The term ping used herein describes a communication message sent between elements. In the preferred embodiment, it is a single communication message, usually designed to fit in a single network communication packet which is sent but does not require an acknowledgement from the receiver. The information can be enveloped and addressed depending on the communication channel utilized, and such details are beyond the scope of this innovation.

Another reason for activation of the monitor routine may be if a notification message was received from the central server (515). If so, then display the notification on the device (525). This may be a message from the administrator telling a user it is their turn, or it may be a message requesting they return to the receptionist, etc. In another embodiment, the message may present the user with a plurality of responses and allow the selected response to be sent back to the administrator.

Another reason for activation of the monitor routine may be a change in Wi-Fi state (517). Wi-Fi state change can indicate movement, or could alter communication option with the central server, so send a ping to the central server (527) designating the change.

Another reason for activation of the monitor routine may be if an alert message was received from the central server (520). An alert message signifies the device is possibly compromised in some manner and causes a recovery mode (549). Other situations may cause a recovery mode (549), such as extended loss of communication signals, location determined to be a ‘danger zone,’ etc. Additionally, in another embodiment, a user may manually cause a recovery mode (549) to draw attention, such as personal emergency, or to force data protection, such as when permanently discarding a device.

Another reason for activation of the monitor routine may be if a message to wipe data was received from the central server (529). This may be because a device is suspected of compromise or may simply be a precautionary activity at the end of a day to protect confidentiality. In this instance, the data clean routines are executed, and the device is disabled (530) or possibly just turned off.

Recovery mode (549) is activated when a device has possibly been removed from designated areas, has lost power or signal, or otherwise is not reporting to the central server. Recovery of the device is facilitated by activating cellular and GPS network communications (550). Next the alarm (sound) is turned on, vibration (movement) is turned on, and the screen (visual) flashes an alert message (553). This is designed to draw attention to the device and reveal its location.

The routine then determines GPS location (557) and logs the time and location (565). Logging may not be necessary but is done to possibly provide additional details of what occurred to the compromised device. Depending on which communication options are available (563), messages may be sent to the central server providing the current location of the device (560).

The process then repeats by turning on the alarm (sound), vibration (movement), and flashing an alert message on the screen (visual) (553). This is in case those alerts have been turned off since they were last turned on. After ‘X’ times through this routine, the value of ‘X’ may be determined and set by an administrator during initial configuration, data clean routines may be executed (555) to delete potentially proprietary or confidential information from the device.

If the ping frequency timer has expired (510) listen for Bluetooth beacons, collect ID and RSSI values for each advertise packet (533). Use RSSI value to determine the distance to the broadcasting source of each beacon. For each recognized ID, get the beacon's location and determine the device's position (535). From the position, identify the current zone, and compare to the previous zone (537).

If the zone has not changed (540), then send a ping to the central server (545). If the zone has change (540), record the current zone, and determine the ping frequency for the new zone (547) before sending a ping to the central server (545). Reset the ping frequency timer (543) and exit the monitor routine (505) to wait for the next activation.

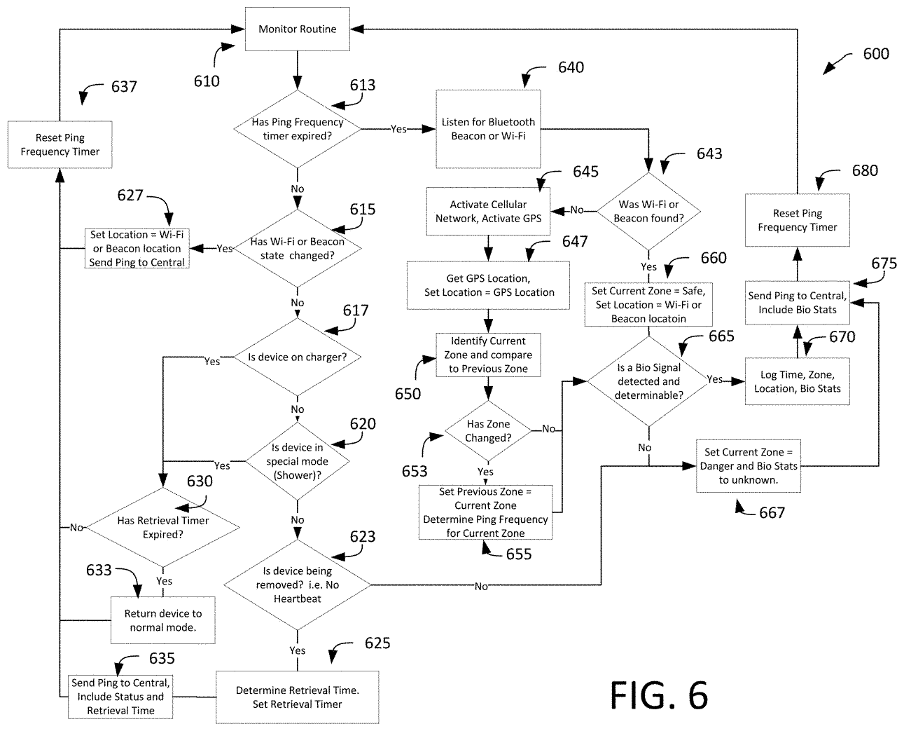

FIG. 6 shows a flowchart of primary operations for a monitoring routine implemented in a wearable device for monitoring and tracking locations and associated data in accordance with an exemplary embodiment of the innovation. A wearable device requires a different monitoring routine (610) because it is used, not to track the device, but to indirectly track the user wearing the device, and to monitor additional characteristics (usually bio readings) of the user. Wearable devices/users may have a larger, less confined, geo fenced area.

The flowchart (600) is for a monitoring routine (610) which is operating in a wearable computing device. The monitoring routine (610) may operate in a continuous looping operation, as illustrated here, or may be a thread in a multi-threaded operation. The thread may be timer-based, rotation-based, or event-based activation. Regardless of the method, each iteration of the routine (610) will need to determine if the ping frequency timer has expired (613).

If the ping frequency timer has expired (613) listen for Bluetooth beacons or Wi-Fi (640). If a recognized Wi-Fi or beacon is found (643), then can set the current location to the Wi-Fi's or beacon's known location, and the current zone to be a ‘safe zone’ (660). The Wi-Fis and/or beacons of ‘safe zones’ are stored along with the geographic location, a user-friendly name, and other data. User-friendly names, and other reference information specific to a tracked-device and/or an administrator will allow the described innovation to formulate user-friendly messages for notifying the administrator(s). Examples include but are not limited to: “Grandma Jones has arrived at Dr's Smith's Office;” “Mr. Davis has left his home.”

If bio signals are detected (665), then the time, zone, location, and bio stats can be logged (670), and sent, as a ping, to the central server (675). The central server can then determine if the information is normal or indicates a reason to alert the administrator(s). One example may be a message to a designated local administrator at an assisted care facility: “Roger Simons, patient 32-4462, has a low-blood sugar reading of 77, and may require assistance. His current location is the Community Center.”

if a recognized Wi-Fi or Bluetooth beacon was not found (643), then activate cellular network, and GPS (645) to determine a device location (647). From the location, the zone can be determined, and compared to the previous zone (650). Note that not being able to receive a GPS signal or a cellular signal is symptomatic of being in an enclosed environment, such as a building, and could be assumed to be the same as the last known location since most enclosed environments are not large enough to be an accuracy issue in locating a person because the interior location is likely a known location, or entirely within a specific zone.

If the zone has changed (653), then record the new zone and determine the ping frequency for the new zone (655). Next, determine bio signals (665) and if detected and determined, log them (670) and send a ping of the data to the central server (675). If bio signals are not detected (665), then set the bio stats to unknown and the current zone to ‘danger zone’ (667) and send the ping to central server (675) to indicate a problem. Reset the ping frequency timer (680) and exit the monitor routine (610) to wait for the next activation.

Upon an activation, if the ping frequency timer has not expired (613), then determine if there is any other reason for activation of the monitor routine. A reason for activation may be a change in beacon or Wi-Fi state (615). Wi-Fi state change can indicate movement or could alter communication option with the central server. Set the location to the Wi-Fi or beacon's location and send a ping to the central server (627).

Another reason for activation of the monitor routine may be a timer monitoring a special state. These are usually initiated from a user input and/or by a user removing the device. I.E. the wearable device is not being worn (623). This can be detected in multiple ways, including, but not limited to, sensing an opening of the clasps, or a loss of bio signals (for instance, no heartbeat detected). If bio stats are not detected, but all indication is that the device has not been removed (623), then send an emergency signal to the central server (675), indicating the loss of bio stats, and a possible emergency situation (667).

If removal is determined (623), we may wait a prescribed amount of time to determine if the device is connected to the charger (617), or placed in another special mode (620) such as delayed readings for a sort time to account for a shower, or medical exam, or swim, etc. Once the reason for removal is determined, the anticipated retrieval time can be determined, and the retrieval timer can be set (625).

A ping is sent to the central server indicating the special mode and the anticipated retrieval time (635) which will indicate when the central server can expect to resume monitoring pings. Special modes allow activity and bio stat to alter or go off-line for a certain amount of time without raising an alarm to the system as previously described above.

Since the device will continue operating the monitor routine (610), It will determine if a special mode is occurring. If the unit is on the charger (617), then a retrieval time would have been established (625) so the device checks if the retrieval timer has expired (630). If the timer has expired, then return the device to normal mode (633) and reset the ping frequency timer (637). Once the device is returned to normal mode, the next activation of the monitor routine (610) will resume normal operation which will raise an alert if the user has not resumed wearing the device.

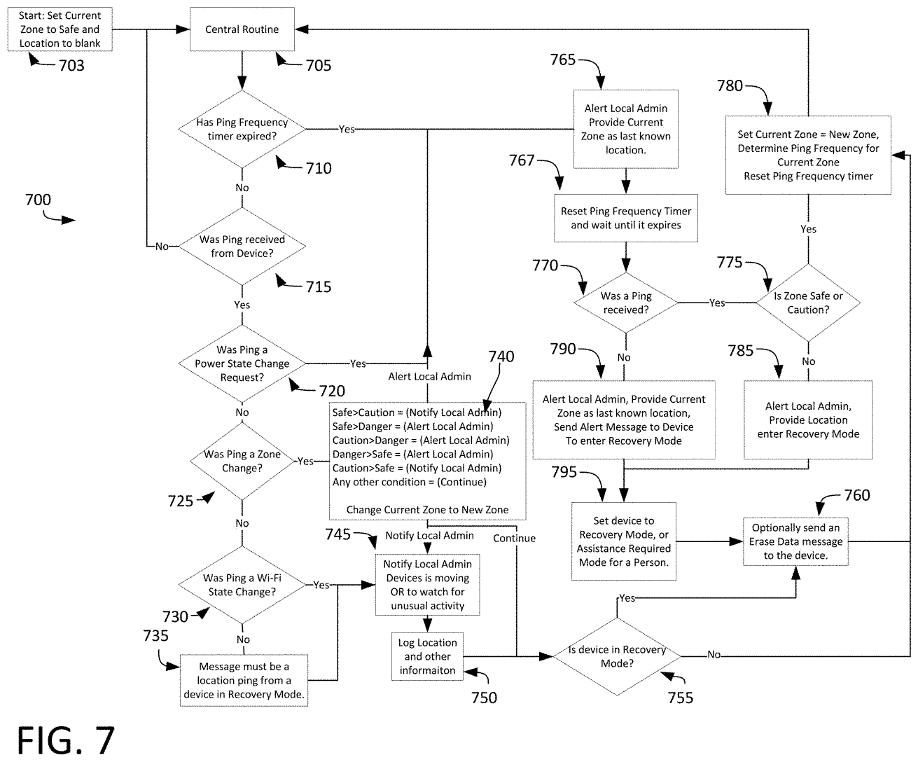

FIG. 7 shows a flowchart of primary operations for a centralized server routine of a monitoring system for monitoring and tracking locations and associated data in accordance with an exemplary embodiment of the innovation. One skilled in the arts would appreciate that the flow/order of operations and the division of operations described herein are not specific, nor all inclusive. Other embodiment may have additional operations or less operations.

The flowchart (700) is for a central server's monitor, which is called a central routine (705) which is may be operating on a computing device capable of multi-threaded operations. The monitoring routine (705), which may also be known as a thread, may be timer-based, rotation-based, or event-based activation. During initial starting of the thread, the zone is set to ‘safe zone,’ and the location is set to ‘unknown’ for all devices being monitored (703). The central routine (705) may monitor a plurality of devices. However, for simplicity, the following description primarily describes monitoring of a single device.

Depending on the method of activation, it may be necessary to determine if the ping frequency timer has expired (710). If it has not expired, then determine if a ping was received from the monitored device (715)? If not, then continue with other processing, such has monitoring other devices. Note that a ping is any communication from a device, as previously described, and would include information provided from a plurality of communication methods.

If the ping frequency timer has expired (710), then the device has not checked in when it was expected. The system alerts the local admin for the device and provides the last known location of the device (765). The routine may optionally allow one or more pings to be missed before alerting (767). This option may be desirable to prevent false triggers particularly in environments with unreliable communications.

One a device is determined to have failed to check in within a specific timeframe (770), the system alerts the local admin for the device, and provide the last known location of the device (790). The system also sends a message to the device triggering the device's recovery mode (795). Setting a device to ‘Recovery Mode’ or ‘Assistance Required Mode’ is an indication that something is not normal, and attention is required. This situation may send a message to the device to erase confidential data (760) or trigger other operations.

If the device does check in within the allowed timeframe (770), then determine if the device's zone is a ‘safe zone’ or a ‘caution zone’ (775), and if so, record the new zone, determine the ping frequency, and reset the ping frequency timer (780). If the device is not in a ‘safe zone’ or a ‘caution zone’ (775), then alert the device's admin with the location, and enter recovery mode (785) as described above.

If the ping frequency has not expired (710), and a ping was received from the device (715), then determine the actions required from the data supplied in the ping. Was the ping providing notice of a power state change request (720)? If so, then process it by alerting the local admin (765) and waiting to see if the device is power cycled and returns to normal operation.

If the ping received was a zone change (725), then determine if the new zone is different from the last zone (740) and depending on the values either A) Alert the local admin (765), B) Notify the local admin (745), or C) Continue processing (755). If the ping received was not a zone change (725), then it may be a Wi-Fi state change (730), in which case the system notifies the device administrator, referred to as a local admin (745).

If the ping was not a Wi-Fi state change (730), then continue processing other types of messages. In the example given, the next option is to assume receipt of a location ping from a device in recovery mode (735). A notification is sent to the local admin with the new device location (745), and the data from the ping is logged (750). If the device sending the ping is in ‘Recovery Mode’ (755), then an ‘Erase Data’ message may be sent back to the device (760) through the same communication method as the received ping in case previous messages were not received.

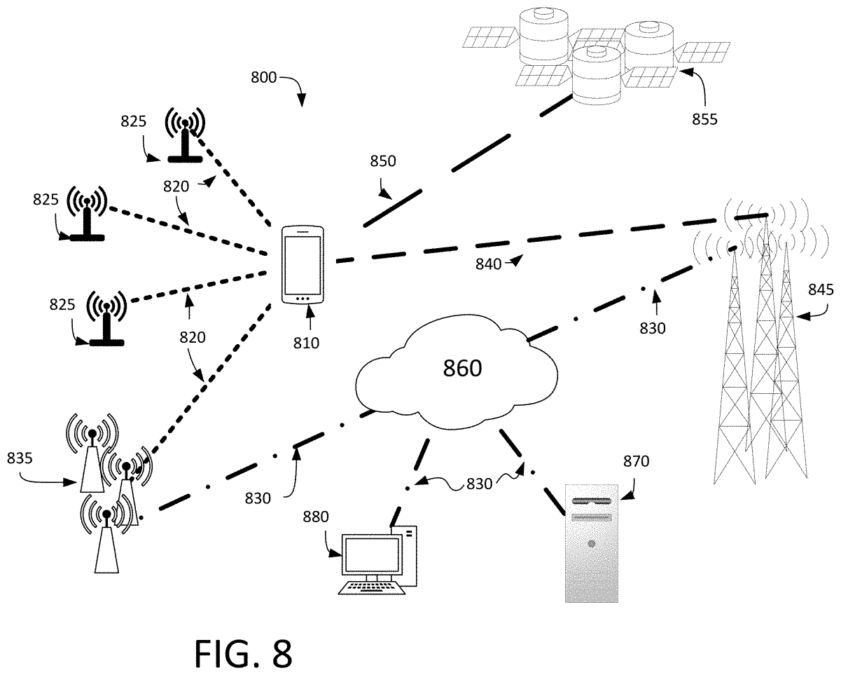

FIG. 8 shows a diagram of communication channel associations for the implementation of monitoring and tracking of locations and associated data in accordance with an exemplary embodiment of the innovation. The communication diagram (800) illustrates a plurality of communications for a tracked device (810). Local wireless communications (820) may be used for a tracked device's (810) transceiver to communicate with beacons (825), or Wi-Fi transceivers (835).

Regional wireless communication (840) may be used for a tracked device (810) to communicate over cellular networks (845) which are connected (830) to WANs (860). Satellite networks (855) can be reached with Broadcast signals (850), through wireless transceivers in the tracked device (810).

Wi-Fi transceivers (835), and some beacons (825) communicate with a local computer network (830), or otherwise connects to the Internet or another Wide Area Network (WAN) (860). A WAN may also allow communications to cellular networks (845), and particularly in this case, with a local admin/administrator (880) and the central manager or central server (870).

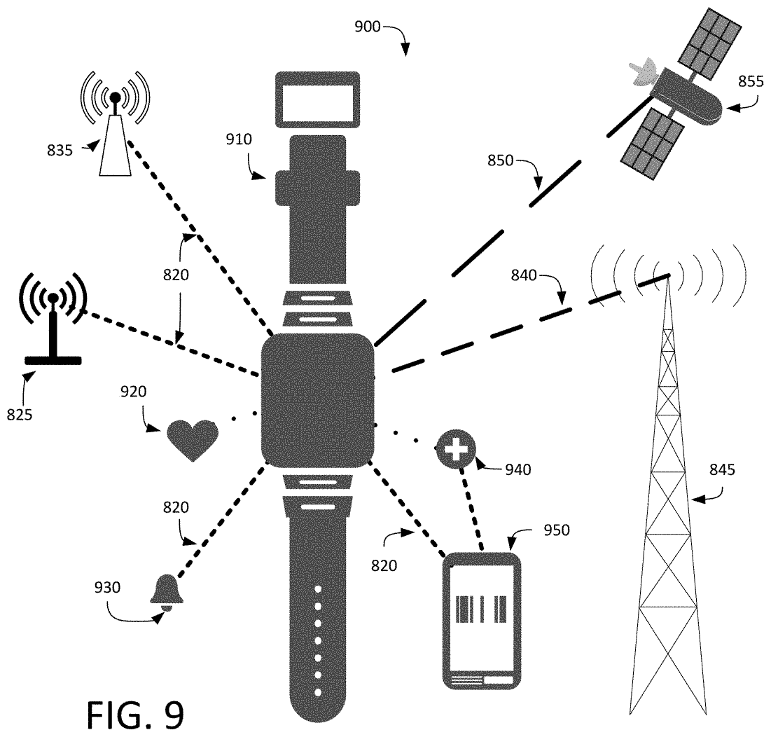

FIG. 9 shows a diagram of communication and sensor requirement for a wearable smart device utilized for implementation of monitoring and tracking of locations and associated data in accordance with an exemplary embodiment of the innovation. The communication diagram (900) illustrates a plurality of communications for a wearable tracked device (910). Local wireless communications (820) may be used for a tracked device's (910) transceiver to communicate with beacons (825), or Wi-Fi transceivers (835). Additionally, regional wireless communication (840) and broadcast signals (850) allow a wearable tracked device (910) to communicate over cellular networks (845) and satellite networks (855), which are connected to WANs (not shown).

Wearable tracked devices (910) can also use local wireless communication (820) to send and receive data for control of exterior devices (930) like, for example, an alarm or intercom. A wearable tracked device (910) may include, or otherwise communicate with a user's biological sensors, such as a heart monitor (920) or an insulin pump (940). These biological monitors may in turn be connected to other smart devices (950) such as a health monitor, that is in communication (820) with a wearable tracked device (910) to provide health monitoring and alerts.

The flow diagrams in accordance with exemplary embodiments of the present innovation are provided as examples and should not be construed to limit other embodiments within the scope of the innovation. For instance, the blocks should not be construed as steps that must proceed in a particular order. Additional blocks/steps may be added, some blocks/steps removed, or the order of the blocks/steps altered and still be within the scope of the innovation.

Further, blocks within different figures can be added to or exchanged with other blocks in other figures. Further yet, specific numerical data values (such as specific quantities, numbers, categories, etc.) or other specific information should be interpreted as illustrative for discussing exemplary embodiments. Such specific information is not provided to limit the innovation.

The diagrams are in accordance with exemplary embodiments of the present innovation and are provided as examples which should not be construed to limit other embodiments within the scope of the innovation. Some elements illustrated in the singularity may actually be implemented in a plurality, and some element illustrated in the plurality could vary in count. Further, some elements illustrated in one form could vary in detail, and other specific information should be interpreted as illustrative for discussing exemplary embodiments. Such specific information is not provided to limit the innovation.

In the various embodiments in accordance with the present innovation, embodiments are implemented as a method, system, and/or apparatus. As one example, exemplary embodiments are implemented as one or more computer software programs to implement the methods described herein. The software is implemented as one or more modules (also referred to as code subroutines, or ‘objects’ in object-oriented programming).

The location of the software will differ for the various alternative embodiments. The software programming code, for example, is accessed by a processor or processors of the computer or server from long-term storage media of some type, such as a CD-ROM drive or hard drive. The software programming code is embodied or stored on any of a variety of known media for use with a data processing system or in any memory device such as semiconductor, magnetic and optical devices, including a disk, hard drive, CD-ROM, ROM, etc.

The code is distributed on such media or is distributed to users from the memory or storage of one computer system over a network of some type to other computer systems for use by users of such other systems. Alternatively, the programming code is embodied in the memory (such as memory of a wearable tracked device) and accessed by the processor using the bus. The techniques and methods for embodying software programming code in memory, on physical media, and/or distributing software code via networks are well known and will not be further discussed herein.

The above discussion is meant to be illustrative of the principles and various embodiments of the present innovation. Numerous variations and modifications will become apparent to those skilled in the art once the above disclosure is fully appreciated. It is intended that the following claims be interpreted to embrace all such variations and modifications.

What is claimed:

1. A method comprising;
monitoring by a central tracker of one or more tracked devices;
the central tracker notifying an administrator of at least one of,
a current location of the one or more tracked devices;
an operation state of the one or more tracked devices;
a change to the location of the one or more tracked devices, or
a change to the operation state of the one or more tracked devices;
wherein each of the one or more tracked devices periodically determines and reports its current location to the central tracker, and each of the one or more tracked devices monitors and reports changes in its operation states and/or sensor values.

2. The method of claim 1 wherein notifying the administrator comprises identifying a specific one of the one or more tracked devices (an identified device), identifying a specific action occurring to the identified device, and optionally, determined by the importance of an action, providing visual and/or audible alerts in addition to textual information of the specific action.

3. The method described in claim 1 wherein the administrator:
selects a specific one of the tracked devices (a specific device);
assigns and provides a user with the specific device, after optionally loading user specific information or user specific configuration of the device;
receiving the specific device from the user;
optionally retrieving and/or removing user specific information and/or restoring a default configuration to the device;
recharging or storing the device for later use by another user.

4. The method described in claim 3 wherein the administrator:
receives a notification from the central tracker;
identifies the user assigned the specific device;
locates the specific device and/or the assigned user; and
retrieves the specific device.

5. The method described in claim 1 wherein monitoring further comprises:
receiving reports from each tracked device;
optionally notifying the administrator of the current location, operation state, and/or sensor values of the tracked device;
recording the current location, operation state, and/or sensor values of the tracked device;
periodically identifying any tracked device from which a report was not received (a missing device); and
notifying an administrator of the missing device.

6. The method described in claim 5 wherein, notifying the administrator of the missing device further comprises:
providing the administrator the recorded current location, operation state, and/or sensor values of the device; and
notifying the missing device to activate a recovery mode.

7. The method described in claim 6 wherein the recovery mode comprises:
optionally locking the device;
optionally deleting confidential and/or user specific information;
activating alarms; and
periodically determine and report the current location of the missing device.

8. The method described in claim 1 wherein determining current location comprises:
receiving one or more communication signals unique to a specific location and assessing a location from the communication signal received.

9. The method described in claim 1 wherein determining current location comprises:
receiving a plurality of communication signals, each having a known origin, and mathematically determining location from the received signal information.

10. The method described in claim 1 wherein determining current location comprises:
receiving three or more communication signals, each having a known transmission rate, and mathematically determining location from the received signal information.

11. A system for location tracking and monitoring of a subject comprising;
the subject physically possessing a tracking device,
the tracking device comprising: a processor, memory, power supply, one or more communication systems, and one or more environmental and/or bio sensors;
the tracking device configured to:
periodically determine and report the current location,
monitor and report changes in one or more sensor values, and/or
monitor and report changes to the tracking device's operating state;
a central tracker, comprising a computing device configured to:
monitor one or more tracked devices, and
send messages to one or more administrators notifying of:
the current location and operation state of a tracked device;
a change in the location of the tracked device,
a change in the operating state of the tracked device, or
a change in one or more sensor values of the tracked device.

12. The system of claim 11 wherein sending messages to one or more administrators further comprises identifying a specific situation or condition of the subject from one or more of the following:
location of the tracked device;
state of the tracked device;
sensor values of the tracked device.

13. The system described in claim 12 wherein monitoring further comprises:
receiving reports from each tracked device;
optionally notifying one or more administrators of the current location, operation state, and/or sensor values of the device;
recording the current location, operation state, and/or sensor values of the device;
periodically identifying any device from which a report was not received (an unknown condition); and
notifying one or more administrators of the unknown condition.

14. The system described in claim 13 wherein, notifying the one or more administrators of the unknown condition further comprises:
notifying one or more emergency service providers of the subject's last known location and condition;
and optionally requesting a physical verification of the subject's condition and/or sensor values.

15. The system described in claim 11 wherein determining the current location comprises:
receiving one or more communication signals unique to a specific location and assessing the tracked device's location from the communication signal received.

16. The system described in claim 11 wherein determining the current location comprises:
receiving a plurality of communication signals, each having a known origin, and mathematically determining the tracked device's location from the received signal information.

17. The system described in claim 11 wherein determining the current location comprises:
receiving three or more communication signals, each having a known transmission rate, and mathematically determining the tracked device's location from the received signal information.
